(19) United States
(12) Patent Application Publication
(10) Pub. No.: US 2011/0124917 A1
(43) Pub. Date: May 26, 2011

(54) PROCESS FOR THE PREPARATION OF CINACALCET AND INTERMEDIATES THEREOF

(75) Inventors: Pietro ALLEGRINI, San Donato Milanese (IT); Emanuele Attolino, Palagiano (IT); Davide Rossi, Malgesso (IT)

(73) Assignee: DIPHARMA FRANCIS s.r.l., Baranzate (IT)

(21) Appl. No.: 12/953,504

(22) Filed: Nov. 24, 2010

(30) Foreign Application Priority Data
Nov. 26, 2009 (IT) MI2009A002079

(51) Int. Cl. C07C 209/28; C07C 209/84

(52) U.S. Cl. 564/373

(57) ABSTRACT

The present invention relates to a process for the preparation of (R)-(1-Naphthalen-1-yl-ethyl)-[3-(3-trifluoromethyl-phenyl)-propyl]-amine or a salt thereof, in particular the hydrochloride, and intermediates useful in its synthesis.

### FIELD OF THE INVENTION

The present invention relates to a process for the preparation of (R)-(1-Naphthalen-1-yl-ethyl)-[3-(3-trifluoromethyl-phenyl)-propyl]-amine or a salt thereof, in particular the hydrochloride, and intermediates useful in its synthesis.

### TECHNOLOGICAL BACKGROUND

Cinacalcet, namely (R)-(1-Naphthalen-1-yl-ethyl)-[3-(3-trifluoromethyl-phenyl)-propyl]-amine of formula (I), is a known compound with anti-hyperparathyroid action, marketed as the hydrochloride.

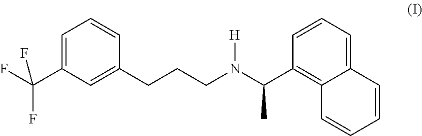

U.S. Pat. No. 6,211,244 discloses its synthesis by condensation of 1-acetyl naphthalene and 3-[3-(trifluoromethyl)phenyl]propylamine in the presence of titanium isopropoxide and subsequent reduction of the resulting imine with sodium cyanoborohydride. The obtained compound (racemic Cinacalcet base) is then resolved by separation of the enantiomers with a chiral chromatographic column.

Drugs of the Future 2002, 27(9), 832 reports a similar process for the preparation of Cinacalcet starting from 3-[3-(trifluoromethyl)phenyl]-propionaldehyde of formula (II)

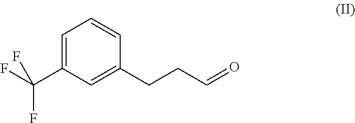

and (R)-1-(1-naphthyl)ethylamine of formula (III)

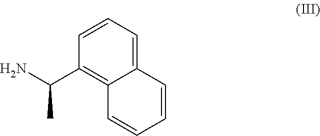

by formation of an imine intermediate and subsequent reduction with sodium cyanoborohydride.

It can be noticed that said processes either make use of toxic reagents, such as sodium cyanoborohydride, or involve the resolution of the racemate, which remarkable affects costs.

On the other hand, WO 06/125026 suggests the synthesis of Cinacalcet of formula (I) by reaction between an alkylating agent of formula (IV) derived from 3-[3-(trifluoromethyl)phenyl]propanol

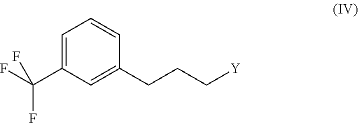

wherein Y is a leaving group, and (R)-1-(1-naphthyl)ethylamine of formula (III).

Said process involves the use of a large excess of alkylating agent (IV) to obtain Cinacalcet free from (R)-1-(1-naphthyl)ethylamine (III), which negatively affects both costs and production times, and is therefore poorly suited to the industrial application.

There is therefore the need for an alternative synthetic route which provides Cinacalcet or a salt thereof in high purity, from low cost starting materials.

### SUMMARY OF THE INVENTION

An alternative process has been found which allows to obtain Cinacalcet or a salt thereof starting from non toxic, inexpensive starting materials and with few synthetic steps. This makes the process of the invention more advantageous than the known processes.

### DETAILED DISCLOSURE OF THE INVENTION

Object of the invention is a process for preparing a compound of formula (I) or a salt thereof

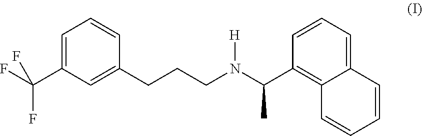

comprising the reductive amination of a compound of formula (II)

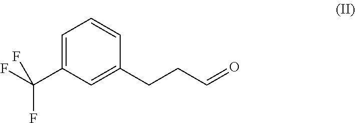

by reaction with (R)-1-(1-naphthyl)ethylamine of formula (III) or a salt thereof

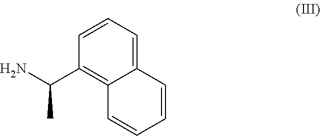

in the presence of a selective reducing agent of the imines.

A selective reducing agent of the imines is for example selected from the group comprising sodium triacetoxyborohydride; the InCl₃/triethylsilane system; the Ti(iPrO)₄/polymethylhydrosiloxane system; Bu₂SnClH; Bu₂SnIH; the PhMe₂SiH/B(C₆F₅)₃ system; Zn(BH₄)₂/silica gel; NiCl₂/NaBH₄ and hydrogen in catalytic hydrogenation conditions; preferably sodium triacetoxyborohydride.

A salt of a compound of formula (I) or (III) is preferably a pharmaceutically acceptable salt, for example the hydrochloride, maleate, sulfate, phosphate, sulfamate, acetate, citrate and tartrate, preferably the hydrochloride salt.

The InCl₃/triethylsilane system can be in the presence of a Fe(II) or Zn(II) salt, for example FeSO₄.

The selective reducing agents of the imines reported above are commercially available and/or known. For example, sodium triacetoxyborohydride is commercially available or can be prepared in situ starting from acetic acid and sodium borohydride.

The catalytic hydrogenation can be carried out with hydrogen and a homogeneous or heterogeneous metal catalyst, based on palladium, platinum, nickel, rhodium or ruthenium, using hydrogen under a pressure which can be comprised between about 1 atm and 20 atm.

When the metal catalyst is heterogeneous, it is preferably deposited on an inert carrier, such as charcoal, barium hydroxide, alumina, calcium carbonate, preferably charcoal. The concentration of the metal on the carrier can be comprised between about 1% and 30%, preferably between 5% and 10%.

The reductive amination is preferably carried out in presence of a solvent selected from a dipolar aprotic solvent, typically dimethylformamide, dimethylacetamide, acetonitrile and dimethylsulfoxide; an ether, typically tetrahydrofuran or dioxane; a straight or branched C₁-C₆ alkanol, preferably a straight or branched C₁-C₄ alkanol such as methanol, ethanol or isopropanol; a C₁-C₆ carboxylic acid such as acetic acid or propionic acid; or a mixture of two or more, preferably two or three, of said solvents; or in water or in an aqueous solution of a protic organic or mineral acid such as trifluoroacetic acid, methanesulfonic and hydrochloric acid, or a mixture thereof with one or more, preferably two or three, of said solvents.

After completion of the reaction, the compound of formula (I) can be recovered as the free base, by means of known methods.

A pharmaceutically acceptable salt of a compound of formula (I) can be obtained by known methods. For example, a salt thereof can be obtained dissolving the free base in ethyl acetate and then reacting it with a protic organic acid, for example acetic, citric, tartaric, fumaric or succinic acid, or with a protic mineral acid, for example hydrochloric, hydrobromic or sulphuric acid, preferably aqueous or gaseous hydrochloric acid.

The recovery of a salt of a compound of formula (I), for example the hydrochloride, can be carried out according to known methods, for example by filtration.

According to a preferred embodiment of the invention, a salt of a compound of formula (I) can be obtained by recrystallization from a solvent in which said salt is poorly soluble at a temperature comprised between about −10° C. and 20° C., whereas the same salt is soluble at a temperature comprised between about 20° C. and the reflux temperature of the crystallization solvent. Typically, a crystallization solvent can be selected from isopropanol, ethyl acetate or an acetonitrile/water mixture.

Cinacalcet hydrochloride, when obtained according to these crystallization conditions, has a XRPD wherein the more intense peaks correspond to those reported in FIG. 1 of WO 06/127933, which is the crystalline form I.

The size of the crystals of a compound of formula (I) or of a salt thereof, obtained according to the invention, is characterized by a D₅₀ value comprised between about 25 and 250 μm. If desired, said value can be reduced by micronisation or fine grinding, according to known methods.

The resulting Cinacalcet hydrochloride has purity equal to or higher than 99.5%, typically equal to or higher than 99.9%.

A compound of formula (III) or a salt thereof is commercially available. An acid addition salt of a compound (III) can be prepared from the corresponding free base by treatment with a protic organic or mineral acid, as defined above, according to known procedures.

Known methods for the preparation of an aldehyde of formula (II)

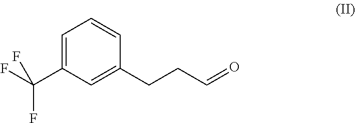

comprise the selective Swern oxidation of the corresponding alcohol (V)

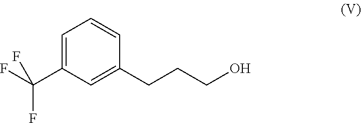

with oxalyl chloride (COCl)₂ and dimethylsulfoxide (DMSO) as reported in Tetrahedron Letters 2004, 45(45), 8355-8358. Said Swern oxidation is a remarkable problem from the industrial point of view, mainly in that it makes use of oxalyl chloride which is unstable and reaction conditions, such as low reaction temperature (generally −60° C.), which are difficult to carry out industrially. An attempt to effect the oxidation of compound of formula (V) to aldehyde (II) was also made using DMSO activated with different activating agents and under temperature conditions more easily reproducible in the industry. The more commonly used DMSO activating agents, such as acetic or trifluoroacetic anhydride and cyanuryl chloride, provided, however, poor results. This route in fact involves formation of too reactive intermediates, even at −10° C., and undesired by-products always form during the oxidation reaction.

There is therefore the need for an alternative process for the preparation of a compound of formula (II), which overcomes the drawbacks reported above and can be carried out on an industrial scale.

It has now been found that oxidation of the hydroxyl function of a compound of formula (V), when carried out with DMSO activated with an activating agent selected for example from P₂O₅, the pyridine/SO₃ complex and dicyclohexylcarbodiimide (DCC), preferably P₂O₅, can be surprisingly and advantageously carried out at a reaction temperature comprised for example between 0° C. and 25° C. The reaction is in fact extremely efficient and cost saving, and the recovery of the product from the reaction mixture is very simple, as no organic by-products are formed, particularly when using P₂O₅.

It is therefore a further object of the invention a process for the preparation of a compound of formula (II) comprising the oxidation of a compound of formula (V) with DMSO activated with an activating agent selected for example from P₂O₅, the pyridine/SO₃ complex and dicyclohexylcarbodiimide (DCC), preferably P₂O₅.

The oxidation reaction can be carried out treating a compound of formula (V) with an at least stoichiometric amount of DMSO and treating the obtained mixture with the DMSO activating agent. The molar ratio of DMSO activating agent to a compound of formula (V) is typically comprised between about 1:1 and 8:1, preferably between about 1:1 and 3:1. The reaction mixture is kept under stirring for a time ranging from about 10 minutes to an hour, then treated with a base and left to react for the time necessary to complete the oxidation, usually less than 10 hours. A base can be organic or inorganic. A base is preferably a tertiary amine, in particular triethylamine, diisopropylethylamine, diazabicycloundecene or diazabicyclooctane.

The oxidation reaction can be carried out at a temperature range comprised between about −10° C. and 50° C., preferably between about 0° C. and 25° C.

The oxidation reaction is preferably carried out in the presence of a solvent, selected from for example a dipolar aprotic solvent, typically dimethylformamide, dimethylacetamide, acetonitrile, dimethylsulfoxide; an ether, typically tetrahydrofuran, dioxane or methyl-tert.butyl ether; a chlorinated solvent, typically dichloromethane; an apolar solvent, typically toluene or hexane; an ester, typically ethyl acetate, isopropyl acetate or butyl acetate; or a mixture of two or more, preferably two or three, of said solvents. The solvent is preferably dichloromethane.

The compound of formula (V) can be obtained by reduction of the triple bond of the compound (VI)

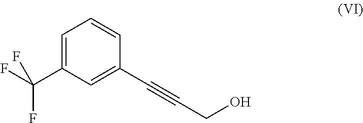

for example by catalytic hydrogenation, by known methods.

The compound (VI) can be prepared from 3-bromo-benzotrifluoride (VII)

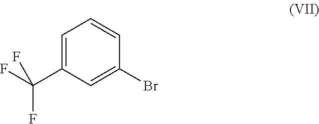

according to known methods, for example according to what reported in EP 2022777.

The following examples illustrate the invention.

### Example 1

### Synthesis of 3-[3-(trifluoromethyl)phenyl]propanol (V)

50 g of 3-bromo benzotrifluoride (0.22 mol, 31 ml) is dissolved in 75 ml of triethylamine and 25 ml of dimethylacetamide under nitrogen atmosphere. The mixture is heated to 50° C., then 340 mg (1.76 mmol) of copper (I) iodide, 155 mg (0.88 mmol) of palladium (II) chloride and 930 mg (3.55 mmol) of triphenylphosphine are added. The mixture is adjusted to 70° C., then 16 ml (16 g, 0.29 mol) of propargyl alcohol is slowly dropped therein. After 17 hours, the reaction mixture is diluted with toluene and filtered. The filtrate is washed in succession with a 1N HCl aqueous solution, a NaHCO₃ aqueous saturated solution and water. The toluene phase is then concentrated under reduced pressure and the resulting compound (VI) is dissolved in methanol (200 ml) and treated with 13.0 g of 5% Pd/C (containing about 49.0% water). The mixture is treated with hydrogen under atmosphere pressure and room temperature for 3 days and subsequently filtered through Celite. The resulting solution is concentrated under reduced pressure to afford 39.5 g of crude compound (V). The resulting compound (V) can be directly used in the subsequent reaction or purified by distillation and recovered as a colourless oil (b.p.: 58-60° C., 1.5 mbars or 82-86° C., 6-7 mbars).

¹H NMR (300 MHz, CDCl₃), ppm: 7.46-7.38 (m, 4H), 3.68 (t, 2H, J 6.3 Hz), 2.78 (t, 2H, J 7.7 Hz), 1.90 (m, 2H).

### Example 2

### Synthesis of 3-[3-(trifluoromethyl)phenyl]-propionaldehyde (II)

A solution of compound (V) (1.0 g, 4.90 mmol) in dichloromethane (20 ml) is cooled in water/ice bath, treated in succession with DMSO (770 mg, 9.80 mmol) and P₂O₅ (1.39 g, 9.80 mmol) and left under stirring for 30 minutes, while temperature raises to 20° C. The reaction mixture is then cooled in water/ice bath and triethylamine (2.4 ml, 17.15 mmol). The resulting solution is kept under stirring while temperature raises to 20° C. After one hour the mixture is treated with 5% HCl, the phases are separated and the organic one is further washed with 5% HCl. The organic phase is then washed with brine, dried over Na₂SO₄, filtered and concentrated under reduced pressure, to afford 0.99 g of the aldehyde of formula (II) in quantitative yield.

¹H NMR (300 MHz, CDCl₃), ppm: 9.83 (t, 1H, J 0.9 Hz), 7.48-7.38 (m, 4H), 3.02 (t, 2H, J 7.2 Hz), 2.82 (m, 2H).

### Example 3

### Synthesis of Compound (I): Cinacalcet Hydrochloride

A solution of compound (II) (1.0 g, 4.95 mmol) and (R)-1-(1-naphthyl)ethylamine hydrochloride (III) (1.03 g, 4.95 mmol) in methanol (10 ml), kept under stirring at 20° C., is treated with solid NaBH(OAc)₃ (2.10 g, 9.89 mmol) in portions. After 2 hours the suspended solid is filtered and the solution is concentrated under reduced pressure. The residue is taken up into toluene and washed first with 1% HCl, then with 5% NaOH. The phases are separated and the organic one is concentrated under reduced pressure to afford a residue substantially consisting of Cinacalcet base.

¹H NMR (300 MHz, CDCl₃), ppm: 8.20 (d, 1H, J 7.5 Hz), 7.88 (m, 1H), 7.76 (d, 1H, J 8.1 Hz), 7.66 (d, 1H, J 7.2 Hz), 7.55-7.43 (m, 5H), 7.38-7.30 (m, 2H), 4.63 (q, 1H, J 6.6 Hz), 2.80-2.56 (m, 4H), 1.84 (m, 2H), 1.50 (d, 2H, J 6.6 Hz).

The residue is then diluted in ethyl acetate and the resulting solution is cooled to 10° C., then hydrochloric acid gas is bubbled therein until marked acidity (pH 2). After one hour, the resulting solid is filtered (1.56 g) to obtain 80% yield and 99% purity. The solid is then redissolved in isopropanol under reflux and recrystallized by slowly cooling the resulting solution. Cinacalcet hydrochloride crystals are washed with water and dried, to afford 1.40 g of product, in 90% yield e purity higher than 99.5%. The resulting product has an XRPD wherein the more intense peaks characterizing correspond to those reported in FIG. 1 of WO 06/127833, corresponding to the form I as defined therein. The size of the crystals is characterized by a D₅₀ value between about 25 and 250 μm.

¹H NMR (300 MHz, DMSO-d₆), ppm: 9.80 (bs, 1H), 9.30 (bs, 1H), 8.22 (d, 1H, J 7.8 Hz), 8.02-7.94 (m, 3H), 7.62-7.44 (m, 7H), 5.28 (q, 1H, J 6.6 Hz), 2.96-2.92 (m, 1H), 2.78-2.66 (m, 3H), 2.04-1.95 (m, 2H), 1.67 (d, 2H, J 6.6 Hz).

1. A method for preparing a compound of formula (I) 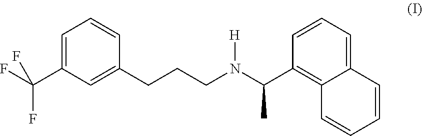
or a salt thereof, which comprises utilizing as staring material a compound of formula (II) 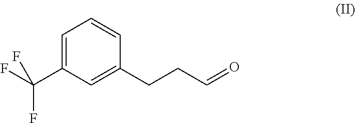
which is obtained by oxidation of a compound of formula (V) 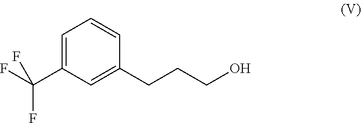
with dimethyl sulphoxide (DMSO) activated with an activating agent other than oxalyl chloride (COCl)₂, acetic anhydride, trifluoroacetic anhydride and cyanuryl chloride.

2. A method according to claim 1 wherein DMSO is activated with an activating agent selected from P₂O₅, the pyridine/SO₃ complex and dicyclohexylcarbodiimide (DCC).

3. A method according to claim 2 wherein the activating agent is P₂O₅.

4. Method according to claim 1, wherein the oxidation reaction is carried out at a temperature comprised between −10° C. and 50° C.

5. Method according to claim 2 wherein the molar ratio between the activating agent of DMSO and a compound of formula (V) is comprised between 1:1 and 8:1.

6. Method according to claim 1, wherein the oxidation reaction is carried out in presence of a solvent, selected from a dipolar aprotic solvent; an ether; a chlorinated solvent; an apolar solvent; an ester and a mixture of two or more of said solvents.

7. Method according to claim 6 wherein the solvent is dichloromethane.

8. Method for preparing a compound of formula (I) or a salt thereof, according to claim 1, further comprising the reductive amination of a compound of formula (II) 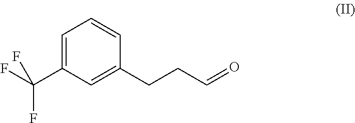
by reaction with (R)-1-(1-naphthyl)ethylamine of formula (III) or a salt thereof 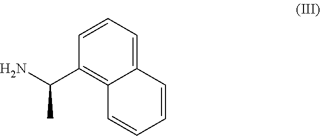
in presence of a selective reducing agent of the imines.

9. Method according to claim 8, wherein the selective reducing agent is selected from sodium triacetoxyborohydride; the InCl₃/triethylsilane system; the Ti(iPrO)₄/polymethylhydrosiloxane system; Bu₂SnClH; Bu₂SnIH; the PhMe₂SiH/B(C₆F₅)₃ system; Zn(BH₄)₂/silica gel; NiCl₂/NaBH₄; and hydrogen in catalytic hydrogenation conditions.

10. Method according to claim 9 wherein the selective reducing agent is sodium triacetoxyborohydride.

11. Method according to claim 8 wherein the reductive amination is carried out in presence of a solvent selected from an aprotic dipolar solvent; an ether; a straight or branched C₁-C₆ alkanol; and a C₁-C₆ carboxylic acid or a mixture of two or more of said solvents; or in water or in an aqueous solution of a protic organic or mineral acid or a mixture thereof with one or more of said solvents.

12. Method according to claim 8 further comprising the preparation of a salt of a compound of formula (I), by reacting a compound of formula (I), as free base, with a protic organic or mineral acid in presence of ethyl acetate.

13. Method according to claim 8 further comprising the recrystallization of a salt of a compound of formula (I), from a solvent in which said salt is poorly soluble at a temperature comprised between −10° C. and 20° C., whereas the same salt is soluble at a temperature comprised between 20° C. and the reflux temperature of the crystallization solvent.

14. Method according to claim 12 wherein the salt of a compound of formula (I) is the hydrochloride salt.

15. Method according to claim 13 wherein the crystallization solvent is selected from isopropanol, ethyl acetate and a mixture acetonitrile/water.
